(19) United States
(12) Patent Application Publication
(10) Pub. No.: US 2009/0047680 A1
(43) Pub. Date: Feb. 19, 2009

(54) METHODS AND COMPOSITIONS FOR HIGH-THROUGHPUT BISULPHITE DNA-SEQUENCING AND UTILITIES

(76) Inventor: SI LOK, Hong Kong (CN)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/192,393

(22) Filed: Aug. 15, 2008

Related U.S. Application Data
(60) Provisional application No. 60/935,472, filed on Aug. 15, 2007.
(60) Provisional application No. 60/935,867, filed on Sep. 5, 2007.

(51) Int. Cl. C12Q 1/68; C12P 19/34

(52) U.S. Cl. 435/6; 435/91.2

(57) ABSTRACT

The invention relates to novel methods and compositions to produce DNA templates suitable for chemical modifications and high-throughput DNA-sequencing. A method of the invention relates to a DNA adaptor design where constituent deoxycytosines are substituted with 5-methyl-deoxycytosines rendering the resulting adaptor resistant to bisulphite mediated deamination. When said adaptor is ligated onto double stranded DNA template, subsequent DNA denaturation and bisulphite treatment deaminates template DNA deoxycytosine differentially to deoxyuraeil whilst the 5-methyl-deoxycytosines of the ligated adaptor resist chemical conversion resulting in the adaptor sequence remaining unaltered. Both strands of bisulphite treated DNA can thus be amplified with a single primer set that hybridizes to the unaltered adaptor sequence. The invention also relates to methods to produce control template of a defined methylation composition to optimize conditions for the bisulphite reaction. In a preferred embodiment, the present invention can be used to produce templates suitable for genome-wide bisulphite-DNA sequencing using conventional, Solexa™, SOLiD™ or 454™-type DNA sequencing platforms to study DNA methylation.

### CROSS REFERENCE TO RELATED APPLICATIONS

This application claims the benefit of U.S. provisional application Ser. Nos. 60/935,472, filed Aug. 15, 2007, and 60/935,867, filed Sep. 5, 2007, which are herein incorporated by reference in their entirety.

### TECHNICAL FIELD AND ABSTRACT OF THE DISCLOSURE

The invention relates to novel methods and compositions to produce DNA templates suitable for chemical modifications and high-throughput DNA-sequencing. A method of the invention relates to a DNA adaptor design where constituent deoxycytosines are substituted with 5-methyl-deoxycytosines rendering the resulting adaptor resistant to bisulphite mediated deamination. When said adaptor is ligated onto double stranded DNA template, subsequent DNA denaturation and bisulphite treatment deaminates template DNA deoxycytosine differentially to deoxyuracil whilst the 5-methyl-deoxycytosines of the ligated adaptor resist chemical conversion resulting in the adaptor sequence remaining unaltered. Both strands of bisulphite treated DNA can thus be amplified with a single primer set that hybridizes to the unaltered adaptor sequence. The invention also relates to methods to produce control template of a defined methylation composition to optimize conditions for the bisulphite reaction. In a preferred embodiment, the present invention can be used to produce templates suitable for genome-wide bisulphite-DNA sequencing using conventional, Solexa™, SOLiD™ or 454™-type DNA sequencing platforms to study DNA methylation.

### BACKGROUND OF THE INVENTION

A major mechanism of epigenetic regulation involves DNA methylation whereby the methyl group of S-adenosyl-methionine is enzymatically transferred to the 5-carbon position of deoxycytosine to yield 5-methyl-deoxycytosine (Review: Caiafa and Zampiere, 2005; Novik et al, 2002; Bird, 2002; Costello and Plass, 2001; Laird and Jaenisch, 1996). In Man, most deoxycytosine methylation occurs at CpG dinucleotides of CpG islands, G+C isochors and CpG hotspots, but deoxycytosine residing in CpNG, CC(a/t)GG, CpA and CpT sequences can also be methylated at low frequency (Lorincz and Groudine, 2001; Woodcock et al, 1997; Clark et al, 1995). Deoxycytosine methylation of CpG dinucleotides in regulatory regions contributes to gene silencing such as in X-chromosome activation and can often play an important role in silencing of tumor suppressor genes in cancers. Hypomethylation and hypermethylation of different genomic regions have been reported at various stages of carcinogenesis as well as in a host of other diseases (Review: Jones and Baylin, 2007; Brena and Costello, 2007; Esteller, 2007; Rodenhiser and Mann, 2006; Laird and Jaenisch, 1996). Hence, there is a need for methods to characterize the “Methylome”, which is defined as the methylation status of the genome, to elucidate regulatory networks that can lead to the discovery of drugs, drug targets, or useful biomarkers of disease.

Various methods have been developed for the analysis of deoxycytosine methylation at an ever-greater resolution to elucidate regulatory networks and for the identification of biomarkers of disease. The earliest method for assaying deoxycytosine methylation was based on chromatography (Hotchkiss, 1948) but it suffered low resolution as this and other related techniques can only look at bulk methylation changes in DNA. Later methods with improved resolution capable of analyzing more precise changes in methylcytosine distribution make use of methyl-sensitive restriction endonucleases or affinity chromatography (Zhang et al, 2006; Cross et al, 1994). Chemical modification of deoxycytosine coupled with DNA sequencing remains the method of choice, the so-termed “Gold Standard”, for the detection of 5-methyl-deoxycytosine at single nucleotide resolution for epigenetic studies. The development of the “Bisulfite-DNA Sequencing” chemistry allows a direct positive detection of deoxycytosine methylation. Bisulphite-DNA sequencing stems from chemistry described in the 1970's, whereby sodium bisulphite catalyzes the efficient deamination of deoxycytosine to yield deoxyuracil (Shapiro et al, 1973; Shapiro et al, 1970; Hayatsu et al, 1970), which is functionally equivalent to deoxythymine in sequencing and DNA amplification reactions. Depending on reaction conditions, the rate for deamination of 5-methyl-deoxycytosine to deoxythymine could be nearly two orders of magnitude slower than that of deoxycytosine to deoxyuracil (Haystsu and Shiragami, 1979; Wang et al, 1980). Frommer et al, (1992) exploited the preferential chemical discrimination between methylcytosine and deoxycytosine in combination with the polymerase chain reaction (PCR) to provide a positive display of 5-methyl-deoxycytosine residing in individual DNA strands. From this initial report and others, bisulphite-DNA sequencing quickly became and remained the method of choice for interrogating deoxycytosine methylation at target loci at single nucleotide level resolution (Ehrich et al, 2007; Grunau et al, 2001; Eads et al, 2000; Paulin et al, 1998; Clark et al, 1994; Raizis et al, 1995; Feil et al, 1994; Frommer et al, 1992).

Attempts are made in recent years to adopt methods for large-scale genome-wide detection of DNA methylation. The first of these attempts is “Restriction Landmark Genomic Scanning” in which differential DNA methylation is detected by serial fractionation of end-labelled DNA digested with methyl-sensitive restriction enzymes (Hatada et al, 1991). This approach is limited by the availability and the distribution of restriction enzyme sites in the genome and is of low resolution. Olek et al, (1998) (U.S. Pat. No 6,214,556) described another approach whereby bisulphite treated DNA is coupled with selective whole genome DNA amplification by the use PCR. Amplified products are then interrogated by primer extension assays to yield complex DNA methylation fingerprints useful for assessing cellular methylation status. The number of primer extension assays performed dictates the resolution and the extent of genomic coverage by this approach. Another strategy is based on affinity purification of methylated DNA segments using anti-methylcytosine antibodies or methyl-CpG binding proteins (Zhang et al, 2006; Cross et al, 1994). Immuno-precipitation and affinity chromatography of methylated Arabidopsis DNA coupled with hybridization of the captured labelled products to a genomic oligonucleotide tiling array has produced the first genome-wide methylation map (Zhang et al, 2006). The resulting methylation map has a 35-base resolution corresponding to the length of the oligonucleotides on the tiling array. Similar studies on human cancer cell lines using arrays of lower resolution have revealed a large number of differentially methylated genes (Keshet et al, 2006; Weber et al, 2005). While useful for genome-wide scan, this approach is hindered by the resolution of the array and by a minimal threshold density of methyl-CpG on a DNA fragment before it can be captured by affinity purification. Accordingly, relatively large amounts of starting materials are needed, thus precluding its use in many clinical applications. Clearly more sensitive detection methods requiring smaller amount of starting material and having higher resolution at the single nucleotide level are needed in the art.

Until recently, technical difficulties have prevented the use of bisulphite-DNA sequencing approach for mapping methylation changes at a genome-wide scale. In a small-scale proof of principle experiment, Meissner et al, (2005) demonstrated the practical feasibility of using bisulphite-DNA sequencing for mapping 5-methyl-deoxycytosines of genomic DNA library inserts at a single nucleotide resolution. Size-selected randomly fragmented genomic DNA fragments, equipped with adaptors were treated with bisulphite, amplified by PCR, and cloned into a vector for sequencing. The resulting sequence data revealed a deoxycytosine to deoxyuracil conversion rate greater than 99.9% indicating that random shotgun bisulphite-DNA sequencing of genomic library inserts could be applied to a genome-wide scale. However, the use of this approach is limited, essentially hindered by the high cost and low throughput of conventional Sanger-based didexoy sequencing and capillary-based electrophoresis. Hence, there remains a need in the art for improved methods of bisulphite-DNA sequencing with lower cost and higher throughput.

The next generation massively parallel DNA sequencing technologies offer several orders of magnitude greater throughput with a corresponding decrease in cost, but as yet, these platforms have not been adapted for bisulphite-DNA sequencing to enable economical genome-wide survey of DNA methylation. There are currently three commercially available systems for high-throughput DNA sequencing: The Genome Sequencer FLX™ system (commonly known as the 454™-sequencer) (Roche Diagnostics, Indianapolis, Ind.); Solexa™ (Illumina, San Diego, Calif.); and the SOLiD™ system (Applied BioSystems, Foster City, Calif.).

The 454-technology is based on conventional pyrosequencing chemistry carried out on clonally amplified DNA templates on microbeads individually loaded onto etched wells of a high-density optical plate (Margulies et al, 2005). Signals generated by each base extension are captured by dedicated optical fibers.

Solexa sequencing templates are immobilized onto a proprietary flow cell surface where they are clonally amplified in situ to form discrete sequence template clusters with densities up to ten-million clusters per square centimeter. Solexa-based sequencing is carried out using primer-mediated DNA synthesis in a step-wise manner in the presence of four proprietary modified nucleotides having a reversible 3′ di-deoxynucleotide moiety and a cleavable chromofluor. The 3′ di-deoxynucleotide moiety and the chromofluor are chemically removed before each extension cycle for successive base calling. Cycles of step-wise nucleotide additions from each template clusters are detected by laser excitation followed by imaging from which base calling is accomplished.

Applied Biosystems' SOLiD approach for massively parallel DNA sequencing is based on sequential of cycles of DNA ligation, a strategy pioneered by George Church of Harvard University (Shendure et al, 2005). By this approach, immobilized DNA templates are clonally amplified on beads (emulsion PCR), which are plated at high density onto the surface of a glass flow cell. Sequence determination is accomplished by successive cycles of ligation of short defined labeled probes onto a series of primers hybridized to the immobilized template.

The throughput from these new instruments can exceed several billion base calls per instrument run, a factor of nearly fifteen thousand-fold or more over the current generation of 96-lane capillary-electrophoresis-based sequencing instruments. Hence, there is an unmet need for methods and compositions to adapt bisulphite-DNA sequencing chemistry to the 454-, Solexa, or SOLiD sequencing platforms to enable cost-effective genome-wide survey of DNA methylation. In their pilot study, Meissner et al, (2005) suggested the new generation 454-DNA sequencer might offer an economical solution to allow genome-wide application of bisulphite-DNA sequencing, but they did not discuss critical problems nor disclose any methods for reduction to practice. More importantly, the investigators did not appreciate the great difficulties in applying their approach to the Solexa or SOLiD platforms where the typical sequence reads are only 35-50 base in length. The present invention provides these and other substantial benefits.

### DESCRIPTION OF THE PRESENT INVENTION

The present invention provides novel improved methods and useful compositions for bisulphite-DNA sequencing for use in next generation DNA sequencers to enable large-scale high throughput genome-wide survey of alterations in deoxycytosine methylation pattern and for other preferred utilities.

The pilot study of Meissner et al, (2005) described a bisulphite-DNA sequencing approach whereby short DNA adaptors are first ligated to each end of a plurality of size-selected and randomly fragmented genomic DNA fragments. Adaptor-ligated DNA is denatured into a single-stranded form that is susceptible to bisulphite treatment where resident deoxycytosines are converted to deoxyuracil but 5-methyl-deoxycytosines are not altered. The converted DNA is amplified using primers to the adaptor region to regenerate the DNA strands and to produce sufficient mass of the bisulphite-converted DNA product for efficient cloning into a vector for sequencing analysis by conventional capillary-electrophoresis. The study shows the approach provides an unbiased representation of the test genomic DNA and has the feasibility of scale. However, an important consequence of Meissner et al's bisulphite treatment of target DNA is that all deoxycytosines in the ligated adaptor are also converted to deoxyuracil. Accordingly, in order to carry out DNA amplification, the PCR primers are designed to hybridize not to the adaptor sequence but are instead designed to hybridize to the bisulphite-converted sequence of the adaptor, the strategy that is the basis of the so termed “Methylation-Specific PCR” method (Cottrell, 2004; Li and Dahlya (2002); Herman and Baylin (1997) (U.S. Pat. No 6,017,704); Herman et al, 1996). Other suitable PCR primer designs known in the art that are suitable to amplify bisulphite treated include the use of degenerate primers that can amplify DNA from bisulphite-modified sites or the use of very short primers that target DNA in deoxycytosine free regions of the DNA (Olek et al, 1998 U.S. Pat. No. 6,214,556).

The restrictions in primer design imposed by methylation-specific PCR as it is described in the art are not compatible for use with the current ABI SOLiD or the Illumina Solexa high-throughput sequencing platforms. These platforms require the obligate use of an optimized and validated proprietary adaptor sequence situated immediately next to the sample DNA insert. These proprietary adaptors function to mediate clonal solid-phase amplification of DNA sequencing templates and the binding of sequencing primers. Read length of the Solexa and SOLiD sequencers is only 35-base (extending to 50-base or more in late 2008). Extraneous sequences situated between the proprietary adaptor and the DNA insert, such as those required for methylation-specific PCR, would reduce the already short read length of the sample DNA to an unacceptable level. Consequently, the current Solexa and SOLiD platforms cannot sequence products produce by the methylation-specific PCR method as it is described (Meissner et al, 2006; Cottrell, 2004; Li and Dahlya, 2002; Hennan and Baylin, 1997, U.S. Pat. No. 6,017,704; Herman et al, 1996). While it may be formally possible to derive an adaptor design in which the sequence of the bisulphite-converted adaptor can mediate clonal amplification on solid support and sequencing primer binding on the Solexa and SOLiD platforms, the technical and economic challenges are formidable. Bisulphite conversion of deoxycytosine to deoxyuracil on the adaptor would effectively reduce the genetic code to only three base, thereby placing the severe constraint on a design that can function efficiently and specifically for solid phase amplification required by the platform and for specific priming of high-throughput DNA sequencing. Moreover, the bisulphite-conversion renders the two strands of the adaptors non-complementary, thereby requiring the creation and validation of an additional set of solid phase amplification primers and sequencing primers for the other sample DNA strand. Considerable company expense, time and resource have been expended to develop and to validate the existing adaptor and primer designs of the SOLiD and Solexa sequencing platforms; a major design change to an existing product already in the marketplace would pose an unacceptable financial burden. Read length of the 454-sequencer is several hundred base and could suffer the reduction of read length imposed by addition of methylation-specific PCR primers in the sample DNA template. However, elimination of extraneous sequences in 454-templates would add to the efficiency of that platform.

The present invention provides novel, simple, effective, and low cost methods to adapt the existing SOLiD, Solexa or 454-based DNA sequencing platforms to sequence bisulphite-treated DNA samples to study DNA methylation. One aspect of the invention is the creation of a novel adaptor composition where constituent deoxycytosines are substituted with 5-methyl-deoxycytosines to render the said adaptor resistant to deamination during bisulphite treatment of the attached template DNA. When adaptor of the present invention is ligated to template DNA, DNA denaturation and bisulphite treatment that convert template DNA deoxycytosine to deoxyuracil, the sequence of the adaptor remains unaltered. Both strands of bisulphite treated DNA can thus be amplified using a single primer set that is complementary to the original altered adaptor sequence. In contrast, deoxycytosines of a conventional adaptor are converted to deoxyuracils by bisulphite treatment necessitating the use of PCR primers that hybridize to the bisulphite-converted sequence of the adaptor to amplify bisulphite treated templates. Bisulphite treatment also renders the two DNA template strands non-complementary. The two strands of a conventional adaptor would also be rendered non-complementary by bisulphite treatment, resulting in the need for a separate set of primers to amplify each DNA strand. The adaptor composition of the present invention does not suffer from this problem, the two adaptor strands remain complementary and a single set of primers is sufficient to amplify both strands of the bisulphite treated DNA for the preparation of templates for sequencing on the Solexa, SOLiD or 454-sequencing platforms. Adoption of present invention by these established platforms is expected to incur little or no material cost since the primary sequence of the platform's propriety adaptor is not altered, hence, all downstream operations such as solid phase DNA amplification and sequencing primer binding are unaffected. In a preferred embodiment, an aspect of the present invention can be used to create kits or kit components for the preparation of DNA templates for high throughput bisulphite-DNA sequencing on the SOLiD, Solexa, 454-, or other sequencing platforms for methylation studies. Kit components are essentially identical to ones currently offered by the vendors for conventional sequencing except for the simple and low cost substitution of 5-methyl-deoxycytosine for deoxycytosine in the adaptors.

Typically, an adaptor comprises two short complementary DNA oligonucleotide strands comprising native or modified oligonucleotides that are produced by chemical or enzyme-assisted synthesis using a variety of synthetic routes known in the art (Review: Verma and Eckstein, 1998; Goodchild, 1990). Oligonucleotides comprising modified bases such as the conjugation of a methyl group at the 5-carbon position of deoxycytosine to yield 5-methyl-deoxycytosine are available from a variety of commercial vendors including: Operon (Cologne, Germany); Sigma-Proligo (Paris, France); and Genosys (St. Louis, Mo.). Alternative to chemical synthesis, it is possible to methylate adaptor DNA enzymatically using methyltransferases providing the deoxycytosines are within the enzyme recognition site. It is also possible to incorporate 5-methyl-dCTP into adaptor DNA by the use of a DNA polymerase in a fill-in reaction or by PCR. Those that are skilled in the art are aware of optimized adaptor designs and the methods of synthesis. Operationally, the two DNA strands of the adaptor are annealed to form a double strand molecule. In general, adaptor sequences may vary from 10 to 100 base pair (bp) or more in length, 15 to 30 bp is typical. Sequence composition of adaptor is variable, but it is generally free of inverted repeats and the like that may interfere with potential primer binding and other functionalities. In some applications, adaptors may be spatially linked together to enable the linked adaptor to ligate to more than one target DNA end. Typical of this application is when it is desirable to have a different adaptor ligated to each end of a template DNA as in the case for clonal amplification and subsequent sequencing on the next generation Solexa, SOLiD or 454-DNA sequencers. Inter-molecular ligation of a linked adaptor to a target DNA is followed by intra-molecular ligation to yield a circular molecule whereby the target DNA is flanked by two different adaptors. Conditions for intramolecular ligation to yield circular molecules have been described for DNA segments over a range of fragment lengths (Collins and Weissman, 1984; Dugaiczyk et al, 1975; Wang and Davidson, 1966). Adaptor may be engineered to have different terminal structures to facilitate ligation to DNA. Blunt-termini are in common use, as are specific cohesive complementary ends for ligation to DNA fragments bearing the partner complementary ends. Procedures for ligation of adaptor to DNA and for genome-wide DNA amplification using primers that target ligated adaptors are known in the art (Hughes et al, 2005; Klein et al, 1999; Lucito et al, 1989; Ludecke et al, 1989; Kinzler and Vogelstein, 1989). Adaptor may comprise other modified or conjugated nucleotides in addition to aforementioned substitution of deoxycytosine with 5-methyl-deoxycytosine. Other chemical modifications of deoxycytosine that can render the adaptor molecule resistant to bisulphite treatment or to other differential chemical treatment that can distinguish genomic deoxycytosine from modified adaptor deoxycytosine are considered within the scope and principle of the present invention. Also considered within the scope and principle of the present invention are modifications of other adaptor bases, in which there are chemical reactions that can distinguish modified adaptor DNA from genomic DNA for use to interrogate other cellular epigenetic DNA modifications. Also considered within the scope and principle of the present invention is the incorporation of an epitope or purification tag to the adaptor, such as a biotin containing moiety or a DNA sequence that can be targeted by a triple-helix forming oligonucleotide (Review: Vasquez and Glazer, 2002; Sun et al, 1996) and the like to allow convenient affinity-purification of the adaptor ligated DNA before, after or during various steps of chemical treatment.

DNA for analysis in accordance to the present invention can be derived from any cell, tissue, or organ. In some embodiments, DNA is derived from a tumor or other cells with a disease phenotype at different time points or stages of clinical treatments to assess the global changes in methylation pattern in the disease state. As such, the present invention can be used to identify genomic diagnostic or prognostic methylation biomarkers of disease or disease susceptibility or disease outcome. Ordway et al, (2006), Sova et al, (2006), and Shames et al, (2006) provide illustrative examples of such biomarkers. Other utilities include the elucidation of regulatory networks that lead to the identification of drugs or drug targets for therapeutic intervention.

DNA for whole-genome methylation study can be generated by random fragmentation to provide an unbiased analysis of the genome. Suitable size DNA may range from 100 to 5000 bp or more, typically 100 to 250 bp is preferred. Methods for generation of random DNA fragments include: (1) bovine pancreatic deoxyribonucleic acid nuclease I (DNase I), which makes random double-strand cleavages in DNA in the presence manganese ions (Melgar and Goldthwait, 1968); (2) physical shearing (Shriefer et al, 1990); and (3) sonication (Deininger, 1983). In some embodiments, genomic DNA may be digested with enzymes that preferentially target digestion to CpG island sequences, which are GC rich regions that are associated with genes in the genome (Kato and Sasaki, 1998). A large proportion of methylation occurs within CpG sequences, hence digestion of genomic DNA with enzymes such as Msp I (CCGG), Hae III (GGCC), Taq I (TCGA) and the like would preferentially target bisulphite-DNA sequencing to those regions of the genome. The use of restriction endonuclease CviJ I under relaxed conditions, which cleaves DNA at GC dinucleotide positions (Fitzgerald et al, 1992), is particularly useful under partial digestion conditions to produce a useful continuum of DNA fragment sizes.

Computer simulation analysis indicates that a given random 50-base read stands a ˜93% chance of an unambiguous assignment to the Human genome reference assembly. For 50-bp fragments flanked by Msp I (CCGG) or Hae III (GGCC) sites and other enzymes that have a G+C rich recognition sites, unambiguous assignment to the genome assembly is greater than 99% due to the observation that most repetitive DNA elements in the genome have lower GC content and that those enzyme sites are under represented in these genomic regions. The computer model also shows a high degree of overlap in fragments generated by the Msp I, Hae III and Taq I digestion. Within the 50-400 bp fragment size range, most CpG island sequences can be covered by overlapping 50-bp reads from a genomic library constructed from individual digestion by the three enzymes. Bisulphite treated DNA generally experiences a lower rate of unambiguous assignment to the reference sequence due to the conversion of deoxycytosine to deoxyuracil (deoxythymine), which effectively reduces the raw query to a three-base genetic code. This problem is manageable using the pair-end read capability of Solexa and SOLiD sequencers to extend the sequence length, and as well as by consensus alignment and contig-building using the opposite DNA strand. In addition to identify changes in methylation status, the present invention would also at the same time identify SNPs and other genetic and somatic alternations when the sequence data are compared to reference sequences. Informatical tools for clustering analysis of methylation data are in the art (Wang et al, 2007; Segal, 2006; Siegmund, 2004; Virmani et al, 2002; Model et al, 2001; Eads et al, 2000).

Despite the usefulness and the widespread use of bisulphite-DNA sequencing, this method is prone to processing errors and the problems of competing and unwanted chemical reactions inherent to bisulphite treatment. These problems will be more pronounced in genome-wide applications. Aggressive bisulphite treatment protocols, (i.e. prolong incubation time, high temperature, or high bisulphite concentration), assure complete conversion of deoxycytosine to deoxyuracil, but risk unacceptable fragmentation of the DNA from depurination as well as the eventual conversion of 5-methyl-deoxycytosine to deoxythymine (Hayatsu and Shiragami, 1979; Wang et al, 1980). Less aggressive treatments run the risk of overestimating methylation levels due to incomplete conversion of deoxycytosine to deoxyuracil. Accordingly, there is a large body of work directed to the continued optimization of the bisulphite conversion process in respect to the major experimental conditions of temperature, pH, reaction time, bisulphite concentration, efficiency of DNA denaturation and the like (Ehrich et al, 2007; Hayatsu et al, 2006; Grunau et al, 2001; Eads et al, 2000; Paulin et al, 1998; Clark et al, 1994; Raizis et al, 1995; Feil et al, 1994; Frommer et al, 1992). A major limiting step for optimizing the process is the lack of a convenient and comprehensive control template to monitor the complex and competing reactions inherent to bisulphite conversion. Current methods for assessing the efficiency of bisulphite conversion make use of high performance liquid chromatography (HPLC), gel electrophoresis, and mass spectrometry to examine the quality of the DNA following treatment (Ehrich et al, 2007). The rate of deoxycytosine conversion to deoxyuracil in bisulphite-reaction optimization experiments is typically measured by methylation-PCR assays, and subsequent sequencing the of cloned product derived from one or more genomic test locci (Frommer et al, 1992) or from a test control template whereby defined sites of both DNA strands are methylated by the use of methyltransferases. Control templates derived from in vitro methylation using methyltransferases suffers from potential incomplete enzymatic action, making it difficult to discern whether the presence of a deoxythymine at a specified site is due to incomplete in vitro methylation or is due to overly aggressive bisulphite conversion in which methylcytosine can be converted to deoxythymine (Hayatsu and Shiragami, 1979; Wang et al, 1980). Moreover, only deoxycytosines that are within the recognition site for a given methyltransferase can be assessed. Hence, there is a need for a convenient, robust and comprehensive assay to monitor the complex and competing reactions in the bisulphite-conversion process, particularly if bisulphite-sequencing is to be carried out at a genome-wide scale.

Another aspect of the present invention provides methods to produce synthetic control templates of a precise defined deoxycytosine methylation composition to optimize the conditions of the bisulphite reaction. In one aspect of the invention, the control template comprises two complementary annealed DNA strands, A and B, wherein the deoxycytosines of strand-A are methylated at the 5-carbon position, and wherein the deoxycytosine of strand-B is not methylated. The resulting hemi-methylated DNA molecule is constructed by annealing the products of two independent amplification reactions derived from a common DNA template. The first reaction comprises amplification primer-A and-B, whereby primer-A deoxycytosines are substituted with 5-methyl-deoxycytosines and primer-B is labeled with a biotin moiety, and amplification is performed in the presence of a deoxyribonucleotide triphosphate mixture comprising dATP, dTTP, dGTP and 5-methyl-dCTP (10 mM of each nucleotide is a typical concentration). The second amplification reaction comprises primer-A and -B, whereby primer-A is labeled with a biotin moiety and amplification is performed in the presence of a deoxyribonucleotide triphosphate mixture comprising of dATP, dTTP, dGTP and dCTP. Equal molar amounts of the two amplified products are combined, denatured, allowed to re-anneal and then are subjected to avidin affinity chromatography to remove DNA molecules that are labeled with biotin. Species not captured by affinity chromatography thus comprise a double-stranded hemimethylated molecule of a methylated deoxycytosine stand-A and an un-methylated deoxycytosine strand-B. The resulting hemimethylated control template (HM-control template) is used to optimize bisulphite reaction conditions. Since the methylation status of the HM-control template is known with absolute precision for each of the two DNA strands, any deviation from the expected sequence or yield of the two control template strands following bisulphite treatment is a quantitative measurement of the degree of incomplete or over aggressive bisulphite treatment. In addition, the control template can be engineered to contain features, such as hair-pins, inverted repeats and the like, that are known to be more resistant to bisulphite treatment to derived experimental conditions to that affect their conversion. In another aspect of the invention, a HM-control template can also be produced by annealing two chemically synthesized oligonucleotides where one strand comprises 5-methyl-deoxycytosines substituting at deoxycytosine positions and the complementary strand comprises deoxycytosine. In another aspect of the invention, a control template can also be generated by PCR in the presence of a deoxyribonucleotide triphosphate mixture comprising dATP, dTTP, dGTP and 5-methyl-dCTP. The resulting control template would have 5-methyl-deoxycytosine completely substituting for deoxycytosine on both DNA strands and is a useful control template to monitor excessive bisulphite treatment. In a preferred embodiment, control templates bearing regions of increasing severity of secondary structure or homo-polymer tracts can be used to monitor the efficiency of bisulphite treatment under different experimental conditions of incubation time, temperature, pH, and bisulphite concentration. In another preferred embodiment, the control template is added to genomic DNA to validate the experimental conditions in the presence of a complex DNA mixture. In another preferred embodiment, a minute amount of the control template can be added to the genomic DNA sample to provide an internal control for high-throughput bisulphite-DNA sequencing on a Solexa, SOLiD or 454-platform. In yet another preferred embodiment, control template of the present invention can be used to provide kits or kit components for high throughput bisulphite-DNA sequencing based on the SOLiD, Solexa, 454-, or other sequencing platforms.

It is to be understood that various other modifications will be apparent to and can readily be made by those that are skilled in the art, given the disclosure herein, without departing from the scope and spirit of this invention.

### REFERENCES

Bird A, 2002. DNA methylation patterns and epigenetic memory. Genes Dev 16: 6-21.
Brena R M and Costello J F, 2007. Genome-epigenome interactions in cancer. Hum Mol Genetics 16: R96-R105.
Caiafa P and Zampieri M, 2005. DNA methylation and chromatin structure: The puzzling CpG islands. J Cell Biochem 94: 257-265.
Clark S J et al, 1995. CpNpG methylation in mammalian cells. Nat Genet 10: 20-27.
Clark S J et al, 1994. High sensitivity mapping of methylated cytosines. Nuc Acids Res 22: 2990-2997.
Collins F S and Weissman S M, 1984. Directional cloning of DNA fragments at a large distance from an initial probe: A circularization method. Proc Natl Acad Sci (USA) 81: 6812-6816.
Cottrell S E, 2004. Molecular diagnostic application of DNA methylation technology. Clin Biochem 37: 595-604.
Costello J F and Plass C, 2001. Methylation matters. J Med Genet 38: 285-303.
Cross S H et al, 1994. Purification of CpG islands using a methylated DNA binding column. Nature Genet 6: 236-244.
Deininger P L, 1983. Random subcloning of sonicated DNA: Application to shotgun DNA sequence analysis. Analyt Biochem 129: 216-223.
Dugaiczyk A et al, 1975. Ligation of Eco RI endonuclease-generated DNA fragments into linear and circular structures. J Mol Biol 96: 171-178.
Eads C A et al, 2000. MethyLight: A high-throughput assay to measure DNA methylation. Nuc Acids Res 28: e32.
Ehrich M et al, 2007. A new method for accurate assessment of DNA quality after bisulphite treatment. Nuc Acids Res 35: e29.
Esteller M, 2007. Epigenetic gene silencing in cancer: The DNA hypermethylome. Hum Mol Gen 16: R50-59.
Feil R et al, 1994. Methylation analysis on individual chromosomes: Improved protocol for bisulphite genomic sequencing. Nuc Acids Res 22: 695-696.
Fitzgerald M C et al, 1992. Rapid shotgun cloning utilizing the two base recognition endonuclease CviJ I. Nuc Acid Res 20: 3753-3762.
Frommer M et al, 1992. A genomic sequencing protocol that yields a positive display of 5-methylcytosine residues in individual DNA strands. Proc Natl Acad Sci (USA) 89: 1827-1831.
Goodchild J, 1990. Conjugates of oligonucleotides and modified oligonucleotides: A review of their synthesis and properties. Bioconjugate Chem 1: 165-187.
Grunau C et al, 2001. Bisulphite genomic sequencing: Systematic investigation of critical experimental parameters. Nuc Acids Res 29: e65.
Hatada I et al, 1991. A genomic scanning method for higher organisms using restriction sites as landmarks. Proc Natl Acad Aci (USA) 88: 9523-9527.
Hayatsu H and Shiragami M, 1979. Reaction of bisulphite with the 5-Hydroxymethyl groups in pyrimidines and in phage DNAs. Biochem 18: 632-637.
Hayatsu H et al, 2006. Does urea promote the bisulphite-mediated deamination of cytosine in DNA? Investigation aiming at speeding-up the procedure for DNA methylation analysis. Nucleic Acids Symposium Series No 50: 69-70.
Hayatsu H et al, 1970. Reaction of sodium bisulphite with uracil, cytosine, and their derivatives. Biochem 9: 2858-2865.
Herman J G and Baylin S B, 1997. Method of detection of methylated nucleic acid using agents which modify unmethylated cytosine and distinguishing modified methylated and unmethylated nucleic acids. U.S. Pat. No. 6,017,704 (Issued Jan. 25, 2000).
Hennan J G et al, 1996. Methylation-specific PCR: A novel PCR assay for methylation status of CpG islands. Proc Natl Acad Aci (USA) 93: 9821-9826.
Hotchkiss R D, 1948. The quantitative separation of purine, pyrimidines, and nucleosides by paper chromatography. J Biol Chem 175: 315-332.
Hughes S et al, 2005. The use of whole genome amplification in the study of human disease. Prog Biophys Mol Biol 88: 173-189.
Jones P and Baylin S B, 2007. The epigenomics of cancer. Cell 128: 683-692.
Kato R and Hiroyuki S, 1998. Quick identification and localization of CpG islands in large genomic fragments by partial digestion of Hpa II and Hha I. DNA Res 5: 287-295.
Keshet I et al, 2006. Evidence for an instructive mechanism of de novo methylation in cancer cells. Nat Genet 38: 149-153.
Kinzler K W and Vogelstein B, 1989. Whole genome PCR: Application in the identification of sequences bound by gene regulatory proteins. Nuc Acids Res 17: 3645-3653.
Klein C A et al, 1999. Comparative genomic hybridization, loss of heterozygosity, and DNA sequence analysis of a single cell. Proc Natl Acad Sci (USA) 96: 4494-4499.
Laird P W and Jaenisch R, 1996. The role of DNA methylation in cancer genetics and epigenetics. Ann Rev Genet 30: 441-464.
Li L-C and Dahlya R, 2002. MethPrimer: Designing primers for methylation PCRs. Bioinformatics 18: 1427-1431.
Lorincz M C and Groudine M, 2001. CmC(a/t)GG methylation: A new epigenetic mark in mammalian DNA? Proc Natl Acad Sci (USA) 98: 11034-10036.
Ludecke H et al, 1989. Cloning defined regions of the human genome by microdissection of banded chromosomes and enzymatic amplification. Nature 338: 248-350.
Lucito R et al, 1998. Genetic analysis using genomics representations. Proc Natl Acad Sci (USA) 95: 4487-4492.
Margulies M et al, 2005. Genome sequencing in microfabricated high-density picrolitre reactors. Nature 437: 376-380.
Meissner A et al, 2005. Reduced representation bisulphite sequencing for comparative high-resolution DNA methylation analysis. Nuc Acids Res 33: 5868-5877.
Melgar E and Goldthwait D A, 1968. Deoxyribonucleic acid nucleases: II. The effect of metals on the mechanism of action of deoxyribonuclease I. J Biol Chem 243: 4409-4416.
Model F et al, 2001. Feature selection for DNA methylation based classification. Bioinformatics 17: S157-S164.
Novik, K L et al, 2002. Epigenomics: Genome-wide study of methylation phenomena. Curr Issues Mol Biol 4: 111-128.
Paulin R et al, 1998. Urea improves efficiency of bisulphite-mediated sequencing of 5′-methylcytosine in genomic DNA. Nuc Acids Res 26: 5009-5010.
Olek et al, 1998. Method for producing complex DNA methylation fingerprints. U.S. Pat. No. 6,214,556 (issued Apr. 10, 2001).
Olek A et al, 1996. A modified and improved method for bisulphite based cytosine methylation analysis. Nuc Acids Res 24: 5064-5066.
Ordway J M et al, 2006. Comprehensive DNA methylation profiling in human cancer genome identifies novel epigenetic targets. Carcinogenesis 27: 2409-2423.
Raizis A M et al, 1995. A bisulphite method of 5-methylcytosine mapping that minimizes template degradation. Anal Biochem 226: 161-166.
Rodenhiser D and Mann M, 2006. Epigentics and human disease: Translating basic biology into clinical applications. CMAJ 174: 341-348.
Schriefer L A et al, 1990. Low pressure DNA shearing: A method for random DNA sequence analysis. Nuc Acids Res 18: 7455.
Segal M R, 2006. Validation in genomics: CpG island methylation revisted. Statistical Applications in Genetics and Molecular Biology 5: article 29.
Siegmund K D et al, 2004. A comparison of cluster analysis methods using DNA methylation data. Bioinformatics 20: 1896-1904.
Shames D S et al, 2006. A genome-wide screen for promoter methylation in lung cancer identifies novel methylation markers for multiple malignancies. PLOS Medicine 3: e486.
Shapiro R et al, 1970. Reactions of uracil and cytosine derivatives with sodium bisulphite: A specific deamination method. J Am Chem Soc 92: 422-424.
Shapiro R et al, 1973. Nucleic acid reactivity and conformation II: Reaction of cytosine and uracil with sodium bisulphite. J Biol Chem 248: 4060-4064.
Shendure J et al, 2005. Accurate multiplex polony sequencing of an evolved bacterial genome. Science 309: 1728-1732.
Sova P et al, 2006. Discovery of novel methylation biomarkers in cervical carcinoma by global demethylation and microarray analysis. Cancer Epidemiol Biomarkers Rev 11: 291-297.
Verma S and Eckstein, 1998. Modified oligonucleotides: Synthesis and strategy for users. Ann Rev Biochem 67: 99-134.
Virmani A K et al, 2002. Hierarchical clustering of lung cancer cell lines using DNA methylation markers. Cancer Epidemiol Biomarkers Rev 11: 291-297.
Wang R Y et al, 1980. Comparison of bisulphite modification of 5-methyldeoxycytidine and deoxycytidine residues. Nuc Acids Res 8: 4777-4790.
Wang Z et al, 2007. Heritable cluster and pathway discovery in breast cancer integrating epigenetic and phenotype data. BMC Bioinformatics 8: 38.
Wang J C and Davidson N, 1966. On the probability of ring closure of lambda DNA. J Mol Biol 19: 469-482.
Weber M et al, 2005. Chromosome-wide and promoter-specific analyses identify sites of differential DNA methylation in normal and transformed human cells. Nat Genet 37: 853-862.
Woodcock D M et al, 1997. Asymmetic methylation in the hypermethylated CpG promoter region of the human L1 retrotransponson. J Biol Chem 272: 7810-7816.
Zhang X et al, 2006. Genome-wide high-resolution mapping and functional analysis of DNA methylation in Arabidopsis. Cell 126: 1189-1201.

1. A method for determining the epigenomic status of a target DNA population comprising:
fragmenting the target DNA population into a suitable size to which one or more different modified-adaptors of a composition comprising a modified nucleotide substituting for its unmodified nucleotide analog at one or more or all positions, are ligated to the target DNA to yield a composition comprising:
(1) an identical modified-adaptor ligated to both ends of the target DNA fragment; or
(2) a different modified-adaptor ligated to each end of the target DNA fragment;
subjecting the modified-adaptor-ligated target DNA to a chemical treatment in which the target DNA composition and the modified-adaptor composition are rendered chemically and functionally distinguishable wherein the ligated modified adaptor or adaptors are essentially and functionally unaltered;
amplifying the chemically treated modified-adaptor-ligated DNA at least one round using primers that are complementary to sequences of the modified adaptor; and
sequencing the amplified DNA.

2. A method according to claim 1, wherein the epigenomic status of the target DNA population is the methylation of deoxycytosine at the 5-carbon position.

3. A method according to claim 1, wherein the one or more modified-adaptors comprise at least one modified nucleotide selected from the group consisting of modified deoxyadenine, modified deoxyguanine, modified deoxycytosine, modified deoxyuracil, and modified deoxythymine nucleotides.

4. A method according to claim 2 wherein the one or more modified-adaptors comprise at least one modified deoxycytosine.

5. A method according to claim 1 wherein the one or more modified-adaptors comprise at least one methylated nucleotide.

6. A method according to claim 5 wherein the methylated nucleotide is 5-methyl-deoxycytosine.

7. A method according to claim 1 wherein the chemical treatment is bisulphite mediated deamination of deoxycytosine.

8. A method according to claim 1, further comprising characterizing the sequence data derived from sources of normal, disease or other phenotypes by mapping or aligning onto one or more reference DNA sequences to discern genetic or epigenomic differences that can be correlated with any phenotypes.

9. A method according to claim 1, wherein the one or more modified-adaptors comprise at least one nucleotide conjugated to a moiety capable of generating a detectable signal that can be read by an instrument or by visual inspection.

10. A method according to claim 1, wherein the one or more modified-adaptors are capable of directing DNA amplification on a solid support.

11. A method according to claim 10, wherein the DNA amplification is isothermal DNA amplification.

12. A method according to claim 1, wherein at least one modified-adaptor is functionally and spatially linked to another modified adaptor.

13. A method according to claim 1, wherein the one or more modified-adaptors comprise at least one nucleotide conjugated to an affinity purification tag.

14. A method according to claim 1, wherein the one or more modified-adaptors comprise at least one nucleotide conjugated to a biotin moiety.

15. A method according to claim 1, wherein the DNA adaptor or adaptors contain one or more sequences that can be targeted by an oligonucleotide capable of forming a triple helix structure with the DNA.

16. A method according to claim 15, wherein the triple-helix-forming oligonucleotide is conjugated to an affinity purification tag.

17. A method according to claim 1, wherein the target DNA is selected from the group consisting of genomic DNA, mitochondrial DNA, chloroplast DNA, plastid DNA, cDNA, viral DNA, microbial DNA, chemically synthesized DNA, DNA product of nucleic acid amplification, and DNA transcribed from RNA.

18. A method according to claim 1, wherein the target DNA is fragmented randomly by the application of mechanical force, or by complete or partial digestion using one or more nuclease enzymes alone or in combination.

19. A method according to claim 18, wherein the nuclease enzymes are restriction endonucleases selected from the group consisting of Bsh1236I, BstUI, CviJI, FspBI, Hae III, Hha I, Hpa II, Mse I, Msp I, Sau3 AI, Taq I, Tsp509 I, and their isoschizomers and neoschizomers.

20. A method for the production of a hemi-methylated DNA control template to monitor bisulphite reaction efficiency comprising:
providing complementary DNA strands, strand-A and strand-B, wherein the deoxycytosines of strand-A are methylated at the 5-carbon position, and wherein the deoxycytosines of strand-B are not methylated; and
annealing a DNA strand-A to a DNA strand-B;
strand-A having been created in a first amplification reaction comprising primer-A and primer-B, whereby primer-A deoxycytosines are substituted with 5-methyl-deoxycytosines and primer-B is labeled with biotin and DNA amplification is performed in the presence of a deoxyribonucleotide triphosphate mixture comprising of dATP, dTTP, dGTP, and 5-methyl-dCTP; and
strand-B having been created in a second amplification reaction using the same template as the first reaction and comprising primer-A and primer-B, whereby primer-A is labeled with biotin and DNA amplification is performed in the presence of a deoxyribonucleotide triphosphate mixture comprising of dATP, dTTP, dGTP and dCTP; and wherein
equal molar amounts of double strand products of the first and the second amplification reactions are combined, denatured, allowed to re-anneal and are then subjected to avidin affinity chromatography to remove any undesired products.

21. A method for the production of a methylated DNA control template wherein the deoxycytosines of both strands are methylated at the 5-carbon position to monitor bisulphite reaction efficiency comprising:
providing a control DNA template; and amplifying that DNA in the presence of a deoxyribonucleotide triphosphate mixture comprising dATP, dTTP, dGTP, and 5-methyl-dCTP using primers where constituent deoxycytosines are substituted with 5-methyl-deoxycytosines.
